(19) United States
(12) Patent Application Publication
(10) Pub. No.: US 2002/0138045 A1
(43) Pub. Date: Sep. 26, 2002

(54) Quick connect fitting

(76) Inventor: Michael E. Moen, Modesto, CA (US)

Correspondence Address: Gene R. Woodle, 3516 Woodle Pl., Rapid City, SD, 57702, US

(21) Appl. No.: 09/814,198

(22) Filed: Mar. 20, 2001

(51) Int. Cl. A61M 5/00; A61M 5/31

(52) U.S. Cl. 604/240; 604/905

(57) ABSTRACT

A quick connect fitting is disclosed which provides a method of connecting a Luer type needle assembly to a syringe body which is quick and easy to use and which provides for a secure and leak proof connection. The quick connect fitting is of the twist on and off type, provides a stop to prevent over tightening of the needle assembly, and also provides a method of securing the needle assembly to prevent accidental loosening or removal.

### FIELD OF THE INVENTION

The present invention relates generally to a quick connect fitting for connecting two hollow bodies together and more particularly to the connection of a Luer type needle assembly to a syringe body.

### BACKGROUND INFORMATION

For many years, thousands of syringes have been used every day for purposes such as the injection of medication and the withdrawal of blood samples. In many cases, the syringe body and a needle assembly, usually known as a Luer type needle or Luer Lock needle, come in two pieces and the needle assembly is connected to the syringe body prior to use. A number of methods of connecting a Luer type needle assembly to a syringe body have been devised. Several problems have arisen in making such connections including: making the connection quick and easy, making the connection secure and leak proof, and making the connection inexpensive to manufacture.

Several attempts have been made to at least partially solve problems relating to connecting a Luer type needle assembly to a syringe body.

An early attempt, the basic design of which is still in wide use, is disclosed in the patent to Dickinson (U.S. Pat. No. 1,742,497; Jan. 15, 1925) which discloses a Luer type needle assembly and a method for connecting the needle assembly to a syringe body. A Luer type needle assembly generally includes a needle at one end and a hub at the other. There is ordinarily a flange with two opposing wings at the base of the hub. The interior of the hub is hollow and in communication with the hollow needle. The Dickinson invention includes a syringe which has a tapered nozzle which fits inside the needle hub. A female threaded collar surrounds the nozzle. The wings of the flange engage the collar threads and, when the needle is turned, the needle is screwed onto the syringe nozzle using the threads.

Another attempt to solve problems relating to connecting a Luer type needle assembly to a syringe body is disclosed in Colsky (U.S. Pat. No. 5,242,401; Sep. 7, 1993). This device also employs Luer type needle assembly having a flange with opposing wings at the base of the hub. The top of the syringe has an opposed pair of “lips” and, when the needle assembly is pressed against the top of the syringe and turned, the flanges are engaged by these lips and held in place by engagement of the bottoms of the lips by the tops of the flanges. Two protruding stops are also provided on the top of the syringe such that the flanges hit the stops and the needle assembly may not be turned passed the point where they engage the lips.

The ideal quick connect fitting provides a method of connecting a Luer type needle assembly to a syringe body which is quick and easy to use and which provides for a secure and leak proof connection. The ideal quick connect fitting should also be simple, lightweight, compact, and inexpensive to manufacture.

### SUMMARY OF THE INVENTION

The present invention employs a conventional Luer type needle assembly with a hollow needle at the forward end and a hub at the rearward end. A pair of opposed flange wings protrude outward from the base of the hub. The interior of the hub is hollow, in communication with the hollow needle, and has the general shape of a truncated cone with the narrow end of the cone being toward the needle.

The rearward portion of the syringe body may be of any appropriate configuration and the forward portion of the syringe body which provides the connection to the Luer type needle assembly is considered a part of the instant invention. For purposes of description, the syringe body is considered to have a left side and a right side which are parallel to the longitudinal axis of the syringe body and a front and back side which are perpendicular to the left and right sides.

A nozzle protrudes forward from the center of the forward end of the syringe body. The nozzle has the same shape as the hollow interior of the hub, but is slightly larger. The nozzle has a hollow, cylindrical core which is in communication with the interior of the syringe body. A pair of syringe flanges are provided which are affixed to the forwardmost surface of the syringe body and are positioned equidistant from the longitudinal axis of the syringe body. Each of the syringe flanges includes a lip which protrudes inward from the forwardmost portion of the syringe flange. The underside of the back syringe flange lip slants downward from left to right. The underside of the front syringe flange lip slants downward from right to left. The left side of the back syringe flange lip is open and the right side of the back syringe flange lip includes a stop which protrudes inward from the right edge of the back syringe flange lip. The right side of the front syringe flange lip is open and the left side of the front syringe flange lip includes a stop which protrudes inward from the left edge of the front syringe flange lip. A nub protrudes downward from the rearward surface of the back syringe flange lip at the right edge of this rearward surface. A nub protrudes downward from the rearward surface of the front syringe flange lip at the left edge of this rearward surface.

In operation, the hollow hub of the needle assembly is placed over the nozzle such that the top surface of the flange wings engage the rearward surface of the syringe flange lips. The needle assembly is turned in a clockwise direction and the slanted undersides of the flange lips cause the needle assembly to be drawn down snugly over the nozzle. The needle assembly is turned until the edges of the flange wings hit the stops and the interior of the hub, the exterior of the nozzle, and the stops are configured such that, at this point, the connection between the needle assembly and the syringe body is tight and leak proof. At this point, the forward surface of the flange wings have engaged and slightly deformed the nubs. The friction and pressure between the nubs and the flange wings secure the needle assembly in position and insure that the needle assembly isn't accidentally dislodge from its secure position. However, by intentionally turning the needle assembly in a counterclockwise direction, the friction and the pressure between the nubs and the flange wings may be easily overcome and the needle assembly removed from the syringe body.

In a second embodiment, the instant invention may be created as a mirror image of that described above such that the needle assembly is connected by turning it counter clockwise and removing the needle assembly by turning it clockwise.

In a third embodiment, the instant invention may be created as described above, but using only a single flange wing and syringe flange.

Although the quick connect fitting of the instant invention has been described above as being used for attaching a needle to a syringe, it could be used as a fitting or connection to connect nearly any of two hollow bodies. One of the major objects of the present invention is to provide a quick connect fitting mechanism for connecting a Luer type needle assembly to a syringe body.

Another objective of the present invention is to provide a quick connect fitting which may be used to connect a needle assembly to the syringe body in a manner which is quick and easy to use.

Another objective of the present invention is to provide a quick connect fitting which provides for a leak proof connection between the needle assembly and the syringe body.

Another objective of the present invention is to provide a quick connect fitting which provides for a secure connection between the needle assembly and the syringe body which connection may not be inadvertently made insecure.

Another objective of the present invention is to provide a quick connect fitting which is simple, lightweight, compact, and inexpensive to manufacture.

These and other features of the invention will become apparent when taken in consideration with the following detailed description and the drawings.

### BRIEF DESCRIPTION OF THE DRAWINGS

FIG. 1 is a perspective view of a preferred form of a quick connect fitting of the present invention;

FIG. 2 is a sectional view of the quick connect fitting taken along line 2-2 of FIG. 1;

FIG. 3 is a top view of the quick connect fitting;

FIG. 4 is detail view of a portion of the quick connect fitting as shown in FIG. 3;

FIG. 5 is detail view of a portion the quick connect fitting as shown in FIG. 2; and

FIG. 6 is detail view of a portion of the quick connect fitting as shown in FIG. 5.

### DESCRIPTION OF A PREFERRED EMBODIMENT

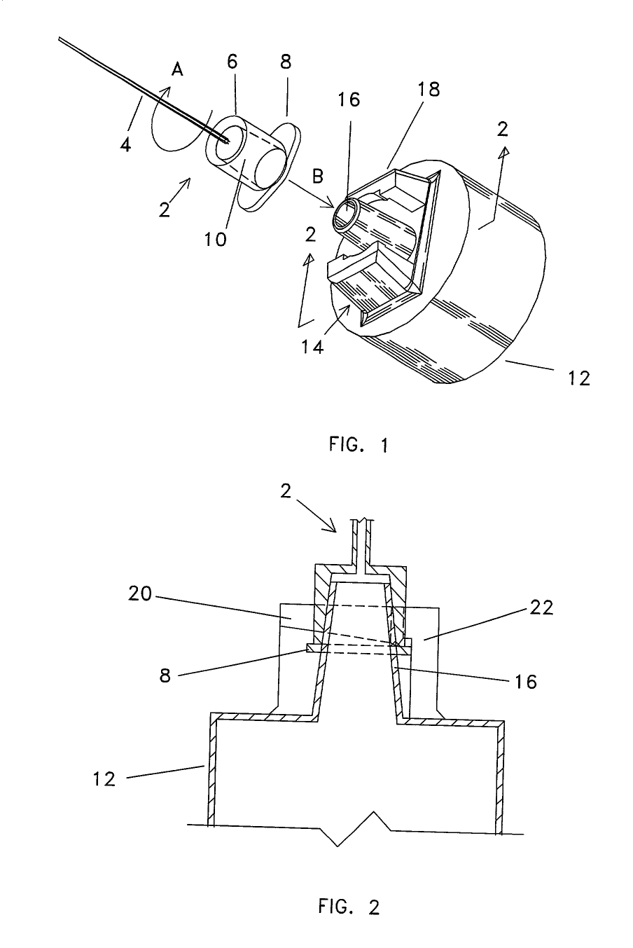

Referring to the drawings, FIGS. 1 through 6, there is shown a preferred form of the quick connect fitting embodying the present invention.

Referring to FIG. 1, there is a needle assembly 2 which is a conventional Luer type needle assembly. The needle assembly 2 includes a needle 4 at its forward end and a hub 6 at its rearward end. A pair of opposed flange wings 8 protrude outward from the base of the hub 6.

The needle 4 is hollow and in communication with the hollow interior 10 of said hub 6. The hollow interior 10 of said hub 6 has the shape of a truncated cone with the smaller end of the cone being toward said needle 4. A syringe body 12 is also provided. The syringe body 12 may have any of a number of useful configurations. A syringe connector assembly 14 is attached to the forward end of said syringe body 12. The syringe connector assembly 14 may be used with any of a number of configurations of said syringe body 12. For purposes of description, said syringe body 12 is considered to have a left side and a right side which are parallel to the longitudinal axis of said syringe body 12 and a front an back side which are perpendicular to the left and right sides. A nozzle 16 protrudes forward from said syringe connector assembly 14. The nozzle 16 is the same shape as said hollow interior 10 and fits within said hollow interior 10. Said nozzle 16 is hollow and is in communication with the interior of said syringe body 12. A pair of syringe flanges 18 also protrude forward from said syringe connector assembly 14. The syringe flanges are equidistant from the longitudinal axis of said syringe body 12. In operation, said needle assembly 2 is pressed onto said nozzle 16 with the flange wings 8 in the openings between the syringe flanges 18 with said nozzle 16 inside said hollow interior 10 as indicated by arrow B. Said needle assembly 2 is then turned clockwise as indicated by arrow A.

Referring now to FIG. 2, which shows a sectional view of the quick connect fitting of the instant invention after said needle assembly 2 is connected to said syringe body 12, said syringe flanges 18 each have a lip 20 which protrudes centrally toward the longitudinal axis of said syringe body 12 from the forward end of said syringe flanges 18. The back syringe flange 18 is shown in FIG. 2 and the lip 20 on the back syringe flange 18 slants rearward from left to right. The front syringe flange 18 is not shown, but the lip 20 on the front syringe flange 18 slants rearward from right to left. When said needle assembly 2 is pressed onto said nozzle 16 and turned as described above, the right, forward face of the back flange wing 8 engages the left, rearward face of said lip 20 of the back syringe flange 18. As said needle assembly 2 is turned as described above, the slanted rearward face of said lip 20 causes said hollow interior 10 to be pulled down snugly over the outer surface of said nozzle 16. Similarly, the slanted rearward face of said lip 20 on said front syringe flange 18 causes an equalizing action on the front flange wing 8. A stop 22 protrudes inward from the right edge of the back syringe flange 18 and from the left edge of the front syringe flange 18. The stop 22 is configured such that when said needle assembly 2 fits snugly over said nozzle 16 the edge of said flange wing 8 hits said stop 22 and said needle assembly is, thus, prevented from turning further.

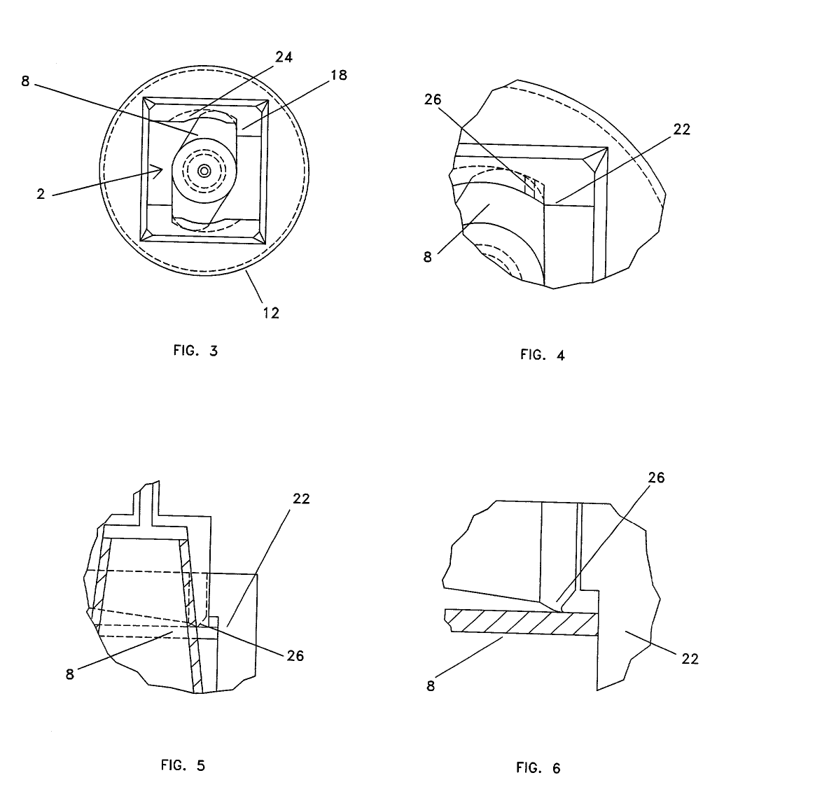

Referring now to FIG. 3, a top view of said needle assembly 2 and said syringe body 12 is shown with said needle assembly 2 connected to said syringe body 12. This view shows that said wing flanges 8 fit within the openings between said syringe flanges 18 when said needle assembly 2 is inserted onto said syringe connector assembly 14. This view also shows the position of said needle assembly 2 at the point at which said wing flanges 8 have hit said stops 22. For ease of manufacture and strength, a cylindrical section 24 is cut away from the interior, rearward portion of each of said syringe flanges 18 to accommodate said flange wings 8 within said syringe flanges 18.

Referring now to FIG. 4, which is a detail view of a portion of the view shown in FIG. 3, there is a nub 26 which protrudes rearward from the rearward surface of said lip 20 adjacent to said stop 22. There is a nub 26 on both the front and rear syringe flanges 18.

Referring now to FIG. 5, which is a detail view of a portion of the view shown in FIG. 2, said nub 26 is shown at the point at which said flange wing 8 is in contact with said stop 22.

Referring now to FIG. 6, a more detailed view showing even greater detail than FIG. 5 is shown. When said needle assembly 2 is turned onto said nozzle 16 as described above and said flange wing 8 hits said stop 22, the forward surface of said flange wing 8 has slightly deformed said nub 26. At this point, the pressure and friction between said flange wing 8 and said nub 26 act to hold said needle assembly 2 in position upon said nozzle 16 creating a secure and leak proof connection between said needle assembly 2 and said syringe body 12. Although said nub 26 acts to hold said needle assembly 2 in place and prevent accidental disengagement of said needle assembly 2 from said syringe body 12; the configuration and manufacture of said nub 26 is such that with positive action a user may easily overcome the pressure and friction between said flange wing 8 and said nub 26 to said needle assembly 2 from said syringe body 12 by turning said needle assembly 2 in a counterclockwise direction. The structure of the rear syringe flange 18 is shown in FIG. 6, and the structure of the front syringe flange 18 is the same.

In operation, the user presses said needle assembly 2 onto said nozzle 16 with said flange wings 8 between said syringe flanges 18 and turns or twists said needle assembly 2 in a clock-wise direction until said flange wings 8 hit said stops 22. Said nubs 26 hold said needle assembly 2 in place and the configuration and manufacture of said syringe connector assembly 14 and said needle assembly 2 cause a secure, leak proof seal between said needle assembly 2 and said nozzle 16 with said needle 4 being in communication with said syringe body 12. The syringe may then be used for any appropriate purpose. The user then may remove said needle assembly 2 from said syringe body 12 by turning or twisting said needle assembly 2 in a counterclockwise direction.

In the preferred embodiment of the quick connect fitting all parts, unless further specified below, are made from a tough, nontoxic, sterilizable plastic sufficiently stiff to provide the necessary rigidity; but sufficiently flexible to allow necessary distortion said nub 26. It is possible that other materials such as aluminum or the like could be used. However, it is necessary that said nozzle 16 be made from a material which is soft or pliable enough to create a seal between said hollow interior 10 of said hub 6 and the outer surface of said nozzle 16. Said needle assembly 2 is made of stainless steel, but other materials could be used provided that they are nontoxic and sterilizable. In the event that the quick connect fitting were to be used for purposes other than for humans or medical reasons, the materials would not have to be nontoxic or sterilizable. As mentioned previously, said syringe body 12 could have any of a number of configurations including the typical handle and plunger assembly.

The quick connect fitting of the instant invention could also be made in a mirror image such that said needle assembly 2 is attached in a counterclockwise direction and removed in a clockwise direction.

In another embodiment, the quick connect fitting of the instant invention could also be made in the same manner, but using a single flange wing 8 and using a single syringe flange 18.

Although the quick connect fitting of the instant invention has been described above as being used to fit a Luer type needle assembly to a syringe body, the fitting could be used to connect and place in communication nearly any hollow, tubular structures including hoses, tubes, etc. In such situations one of two tubular structures would have a male end such as said syringe body 12 described above with a version of said nozzle 16 etc. and the other tubular structure would have a female end which would include an element such as said hub 6, said flange wings 8, and said hollow interior 10. The two tubular structures would then be connected and released in the same manner as described above.

While preferred embodiments of this invention have been shown and described above, it will be apparent to those skilled in the art that various modifications may be made in these embodiments without departing from the spirit of the present invention. For that reason, the scope of the invention is set forth in the following claims:

I claim:

1. A quick connect fitting for attaching a Luer type needle assembly having a hollow needle attached to a hub, the hub having two flanges protruding from the base of the hub opposite each other and said hub being hollow with the shape of a truncated cone tapering in the direction of the hollow needle and the interior of said hub being in communication with the interior of the needle to the end of a syringe body which has a conventional rearward end, a forward end, a right side, a left side, a top, and a bottom:
(1) a nozzle having the same shape as the interior of said hub and being slightly larger than the interior of said hub such that said hub fits snugly over said nozzle but the base of said hub does not reach the base of said nozzle, said nozzle being hollow, attached to the forward end of the syringe body, and being in communication with the interior of said syringe body;
(2) a pair of syringe flanges attached to the forward end of said syringe body and arrayed opposite one another on either side of said nozzle, the syringe flanges being in the shape of an arc circumscribed about the longitudinal axis of said nozzle and the distance between the interior surfaces of said syringe flanges being slightly greater than the distance between the outermost surfaces of the two flanges on said hub; and
(3) a pair of lips attached to and protruding inward from the forward ends of the two syringe flanges, the distance between the inner surfaces of the lips being less than the distance between the outermost surfaces of the two flanges on said hub and being greater than the largest outside diameter of said hub; and the rearward surface of said lips slanting rearward in the clockwise direction such that when said hub is placed over said nozzle and the forward surfaces of said flanges on said hub engage the rearward surfaces of said lips and the needle assembly is turned in a clockwise direction the slanting surfaces of said lips cause said needle assembly to be pulled rearward until the interior surface of said hub snugly contacts the exterior surface of said nozzle;
Whereby a Luer type needle assembly may be quickly, securely, and removably attached to a syringe body by placing the needle assembly over a nozzle upon the end of the syringe body, engaging the forward surfaces of the flanges on the needle assembly with rearward surfaces of lips upon syringe flanges protruding forward from the forward surface of the syringe body and rotating the needle assembly in a clockwise direction.

2. A quick connect fitting of claim 1 in which two stops are provided which protrude inward from the furthest clockwise edge of said syringe flanges such that after said needle assembly has been twisted clockwise onto said nozzle and the interior of said hub of said needle assembly fits snugly against the exterior of said nozzle, the edges of said flanges hit the two stops and prevent said needle assembly from being rotated further clockwise.

3. A quick connect fitting of claim 1 in which two nubs are provided which protrude rearward from the furthest clockwise edge of the rearward surface of said lips and, after the interior of said hub of said needle assembly fits snugly against the exterior of said nozzle, the two knobs press against the forward surface of said flanges and hold said needle assembly firmly in place.

4. A quick connect fitting of claim 2 in which two knobs are provided which protrude rearward from the furthest clockwise edge of the rearward surface of said lips and, after the interior of said hub of said needle assembly fits snugly against the exterior of said nozzle, the two knobs press against the forward surface of said flanges and hold said needle assembly firmly in place.

5. A quick connect fitting of claim 1 in which a single syringe flange with a single attached lip is supplied.

6. A quick connect fitting of claim 2 in which a single syringe flange with a single attached lip is supplied.

7. A quick connect fitting of claim 3 in which a single syringe flange with a single attached lip is supplied.

8. A quick connect fitting of claim 4 in which a single syringe flange with a single attached lip is supplied.

9. A quick connect fitting for attaching a Luer type needle assembly having a hollow needle attached to a hub, the hub having two flanges protruding from the base of the hub opposite each other and said hub being hollow with the shape of a truncated cone tapering in the direction of the hollow needle and the interior of said hub being in communication with the interior of the needle to the end of a syringe body which has a conventional rearward end, a forward end, a right side, a left side, a top, and a bottom:
(1) a nozzle having the same shape as the interior of said hub and being slightly larger than the interior of said hub such that said hub fits snugly over said nozzle but the base of said hub does not reach the base of said nozzle, said nozzle being hollow, attached to the forward end of the syringe body, and being in communication with the interior of said syringe body;
(2) a pair of syringe flanges attached to the forward end of said syringe body and arrayed opposite one another on either side of said nozzle, the syringe flanges being in the shape of an arc circumscribed about the longitudinal axis of said nozzle and the distance between the interior surfaces of said syringe flanges being slightly greater than the distance between the outermost surfaces of the two flanges on said hub; and
(3) a pair of lips attached to and protruding inward from the tops of the two syringe flanges, the distance between the inner surfaces of the lips being less than the distance between the outermost surfaces of the two flanges on said hub and being greater than the largest outside diameter of said hub; and the rearward surface of said lips slanting rearward in the counterclockwise direction such that when said hub is placed over said nozzle and the forward surfaces of said flanges on said hub engage the rearward surfaces of said lips and the needle assembly is turned in a counterclockwise direction the slanting surfaces of said lips cause said needle assembly to be pulled rearward until the interior surface of said hub snugly contacts the exterior surface of said nozzle;
Whereby a Luer type needle assembly may be quickly, securely, and removably attached to a syringe body by placing the needle assembly over a nozzle upon the end of the syringe body, engaging the forward surfaces of the flanges on the needle assembly with rearward surfaces of lips upon syringe flanges protruding forward from the forward surface of the syringe body and rotating the needle assembly in a counterclockwise direction

10. A quick connect fitting of claim 9 in which two stops are provided which protrude inward from the furthest counterclockwise edge of said syringe flanges such that after said needle assembly has been twisted counterclockwise onto said nozzle and the interior of said hub of said needle assembly fits snugly against the exterior of said nozzle, the edges of said flanges hit the two stops and prevent said needle assembly from being rotated further counterclockwise.

11. A quick connect fitting of claim 9 in which two knobs are provided which protrude rearward from the furthest counterclockwise edge of the rearward surface of said lips and, after the interior of said hub of said needle assembly fits snugly against the exterior of said nozzle, the two knobs press against the forward surface of said flanges and hold said needle assembly firmly in place.

12. A quick connect fitting of claim 10 in which two knobs are provided which protrude rearward from the furthest counterclockwise edge of the rearward surface of said lips and, after the interior of said hub of said needle assembly fits snugly against the exterior of said nozzle, the two knobs press against the forward surface of said flanges and hold said needle assembly firmly in place.

13. A quick connect fitting of claim 9 in which a single syringe flange with a single attached lip is supplied.

14. A quick connect fitting of claim 10 in which a single syringe flange with a single attached lip is supplied.

15. A quick connect fitting of claim 11 in which a single syringe flange with a single attached lip is supplied.

16. A quick connect fitting of claim 12 in which a single syringe flange with a single attached lip is supplied.

17. A quick connect fitting for connecting two hollow bodies, one of the hollow bodies having a female end and the other hollow body having a male end, the female end being the forward portion of the fitting and the male end being the rearward portion of the fitting:
(1) the female end having a hollow hub being in communication with the hollow interior of the body and having the interior shape of a truncated cone with the smaller end of the cone being at the forward end of the hub;
(2) two flanges protruding outward from the base of said hub at the rearward end of said hub and arrayed opposite from one another;
(3) the male end having a nozzle, the nozzle having the same shape as the interior of said hub and being slightly larger than the interior of said hub such that said hub fits snugly over said nozzle but the base of said hub does not reach the base of said nozzle, said nozzle being hollow, attached to the forward end of said male end, and being in communication with the interior of the hollow body;
(2) a pair of male end flanges attached to the forward end of said male end and arrayed opposite one another on either side of said nozzle, the male end flanges being in the shape of an arc circumscribed about the longitudinal axis of said nozzle and the distance between the interior surfaces of said male end flanges being slightly greater than the distance between the outermost surfaces of the two flanges on said hub; and
(3) a pair of lips attached to and protruding inward from the tops of the two male end flanges, the distance between the inner surfaces of the lips being less than the distance between the outermost surfaces of the two flanges on said hub and being greater than the largest outside diameter of said hub; and the rearward surface of said lips slanting rearward in the clockwise direction such that when said hub is placed over said nozzle and the forward surfaces of said flanges on said hub engage the rearward surfaces of said lips and the female end is turned in a clockwise direction the slanting surfaces of said lips cause said female end to be pulled rearward until the interior surface of said hub snugly contacts the exterior surface of said nozzle;
Whereby a female end of a hollow body may be quickly, securely, and removably attached to a male end of another hollow body by placing the female end over a nozzle upon the end of the male end, engaging the forward surfaces of the flanges on the female end with rearward surfaces of lips upon male end flanges protruding forward from the forward surface of the male end and rotating the female end in a clockwise direction.

18. A quick connect fitting of claim 17 in which two stops are provided which protrude inward from the furthest clockwise edge of said male end flanges such that after said female end has been twisted clockwise onto said nozzle and the interior of said hub of said female end fits snugly against the exterior of said nozzle, the edges of said flanges hit the two stops and prevent said female end from being rotated further clockwise.

19. A quick connect fitting of claim 17 in which two nubs are provided which protrude rearward from the furthest clockwise edge of the rearward surface of said lips and, after the interior of said hub of said female end fits snugly against the exterior of said nozzle, the two knobs press against the forward surface of said flanges and hold said female end firmly in place.

20. A quick connect fitting of claim 18 in which two nubs are provided which protrude rearward from the furthest clockwise edge of the rearward surface of said lips and, after the interior of said hub of said female end fits snugly against the exterior of said nozzle, the two knobs press against the forward surface of said flanges and hold said female end firmly in place.

21. A quick connect fitting of claim 17 in which a single male end flange with a single attached lip is supplied.

22. A quick connect fitting of claim 18 in which a single male end flange with a single attached lip is supplied.

23. A quick connect fitting of claim 19 in which a single male end flange with a single attached lip is supplied.

24. A quick connect fitting of claim 20 in which a single male end flange with a single attached lip is supplied.

25. A quick connect fitting for connecting two hollow bodies, one of the hollow bodies having a female end and the other hollow body having a male end, the female end being the forward portion of the fitting and the male end being the rearward portion of the fitting:
(1) the female end having a hollow hub being in communication with the hollow interior of the body and having the interior shape of a truncated cone with the smaller end of the cone being at the forward end of the hub;
(2) two flanges protruding outward from the base of said hub at the rearward end of said hub and arrayed opposite from one another;
(3) the male end having a nozzle, the nozzle having the same shape as the interior of said hub and being slightly larger than the interior of said hub such that said hub fits snugly over said nozzle but the base of said hub does not reach the base of said nozzle, said nozzle being hollow, attached to the forward end of said male end, and being in communication with the interior of the hollow body;
(2) a pair of male end flanges attached to the forward end of said male end and arrayed opposite one another on either side of said nozzle, the male end flanges being in the shape of an arc circumscribed about the longitudinal axis of said nozzle and the distance between the interior surfaces of said male end flanges being slightly greater than the distance between the outermost surfaces of the two flanges on said hub; and
(3) a pair of lips attached to and protruding inward from the tops of the two male end flanges, the distance between the inner surfaces of the lips being less than the distance between the outermost surfaces of the two flanges on said hub and being greater than the largest outside diameter of said hub; and the rearward surface of said lips slanting rearward in the counterclockwise direction such that when said hub is placed over said nozzle and the forward surfaces of said flanges on said hub engage the rearward surfaces of said lips and the female end is turned in a counterclockwise direction the slanting surfaces of said lips cause said female end to be pulled rearward until the interior surface of said hub snugly contacts the exterior surface of said nozzle;
Whereby a female end of a hollow body may be quickly, securely, and removably attached to a male end of another hollow body by placing the female end over a nozzle upon the end of the male end, engaging the forward surfaces of the flanges on the female end with rearward surfaces of lips upon male end flanges protruding forward from the forward surface of the male end and rotating the female end in a counterclockwise direction.

26. A quick connect fitting of claim 25 in which two stops are provided which protrude inward from the furthest counterclockwise edge of said male end flanges such that after said female has been twisted counterclockwise onto said nozzle and the interior of said hub of said female end fits snugly against the exterior of said nozzle, the edges of said flanges hit the two stops and prevent said female end from being rotated further counterclockwise.

27. A quick connect fitting of claim 25 in which two nubs are provided which protrude rearward from the furthest counterclockwise edge of the rearward surface of said lips and, after the interior of said hub of said female end fits snugly against the exterior of said nozzle, the two knobs press against the forward surface of said flanges and hold said female end firmly in place.

28. A quick connect fitting of claim 26 in which two nubs are provided which protrude rearward from the furthest counterclockwise edge of the rearward surface of said lips and, after the interior of said hub of said female end fits snugly against the exterior of said nozzle, the two knobs press against the forward surface of said flanges and hold said needle female end in place.

29. A quick connect fitting of claim 25 in which a single male end flange with a single attached lip is supplied.

30. A quick connect fitting of claim 26 in which a single male end flange with a single attached lip is supplied.

31. A quick connect fitting of claim 27 in which a male end syringe flange with a single attached lip is supplied.

32. A quick connect fitting of claim 28 in which a single male end flange with a single attached lip is supplied.
